(19) United States
(12) Plant Patent Application Publication
(10) Pub. No.: US 2007/0163015 P1
(43) Pub. Date: Jul. 12, 2007

(54) Raspberry plant name 'Driscoll Sevillana'

(76) Inventors: Carlos Fear, Aptos, CA (US); Rick Harrison, Aptos, CA (US)

Correspondence Address: JONES DAY, 222 EAST 41ST ST, NEW YORK, NY, 10017, US

(21) Appl. No.: 11/327,110

(22) Filed: Jan. 6, 2006

(51) Int. Cl. A01H 5/00

(52) U.S. Cl. PLT/204

(57) ABSTRACT

The present invention relates to a new and distinct cultivar of raspberry plant named ‘Driscoll Sevillana’. The new cultivar is distinguished from other raspberry cultivars by its large firm fruit with consistent fruit structure and high yield. The new cultivar is distinguished from its parent by being a less vigorous plant, producing more uniform and more elongated shape fruit, and having larger and less acidic fruit.

### 1. LATIN NAME OF THE GENUS AND SPECIES OF THE PLANT CLAIMED

The variety is botanically identified as Rubus idaeus L. subgenus Rubus.

### 1.2 VARIETY DENOMINATION

The Raspberry variety denomination is ‘Driscoll Sevillana’.

### 2. BACKGROUND OF THE INVENTION

This invention relates to a new cultivar of raspberry called ‘Driscoll Sevillana’. The new cultivar was developed from the hybridization of the selection ‘Isabel’ (U.S. Pat. No. PP 9,340) as the seed parent with the selection ‘Driscoll Cardinal’ (U.S. Pat. No. PP 14,903) as the pollen parent. The parents were crossed in 1998, whereafter fruit and seed were collected to produce seedlings for field planting in Oxnard, Calif. in 1999. The new cultivar was selected from these seedlings in 1999 for its large firm fruit. The new cultivar has been asexually propagated by in vitro shoot tip culture, root sucker division and root cuttings at the Cassin Ranch in Santa Cruz County, Calif. and has been shown to maintain the desired and distinguishing characteristics after propagation over several generations.

### 3. SUMMARY OF THE INVENTION

The present invention provides a new and distinct cultivar of red raspberry plant named ‘Driscoll Sevillana’. The variety is botanically identified as Rubus idaeus L. The ‘Driscoll Sevillana’ red raspberry plant produces a primocane crop which begins in mid July and continues until early November. The floricane crop begins in late April and continues until late June. Both the primocane and floricane yields are high relative to other comparable varieties. The fruit of ‘Driscoll Sevillana’ is notably quite firm and very consistent with regard to its size and shape throughout its harvest period. The fruit of ‘Driscoll Sevillana’ separates easily from its receptacle.

### 4. BRIEF DESCRIPTION OF THE DRAWINGS

The accompanying photographs show typical specimens of the primocane fruit, leaves and shoot of the new cultivar, in color as nearly true as it is reasonably possible to make in color illustrations of these characteristics.

FIG. 1 is a photograph of ‘Driscoll Sevillana’ primocane flowers and fruit in various stages of development.

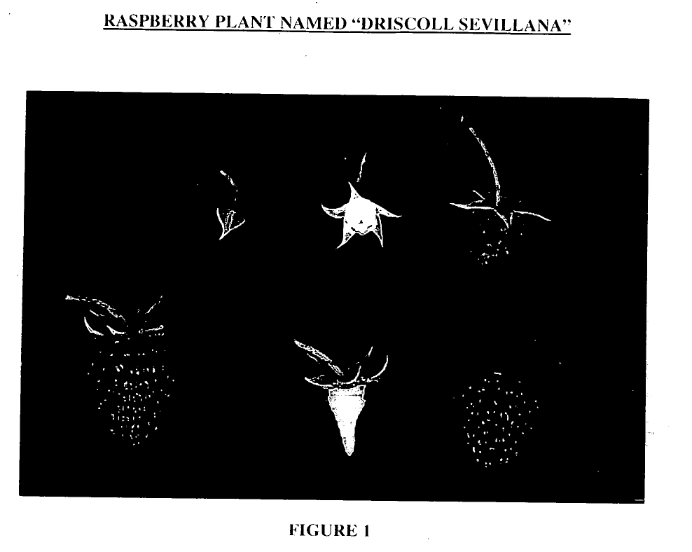

FIG. 2 is a photograph of ‘Driscoll Sevillana’ primocane leaves showing upper and lower leaf surfaces.

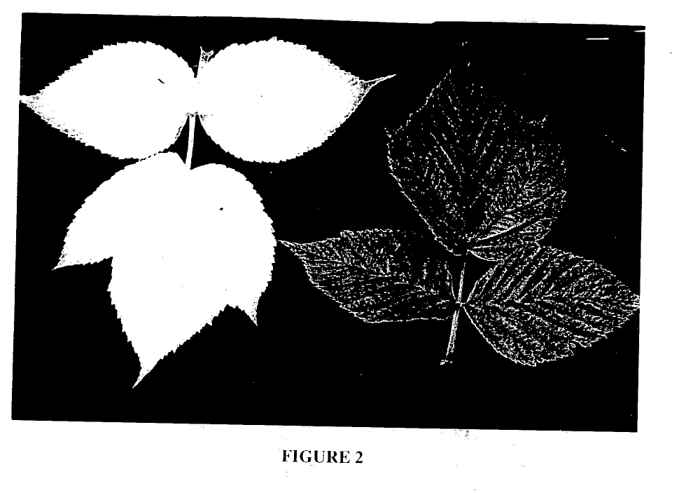

FIG. 3 is a photograph of a ‘Driscoll Sevillana’ primocane shoot.

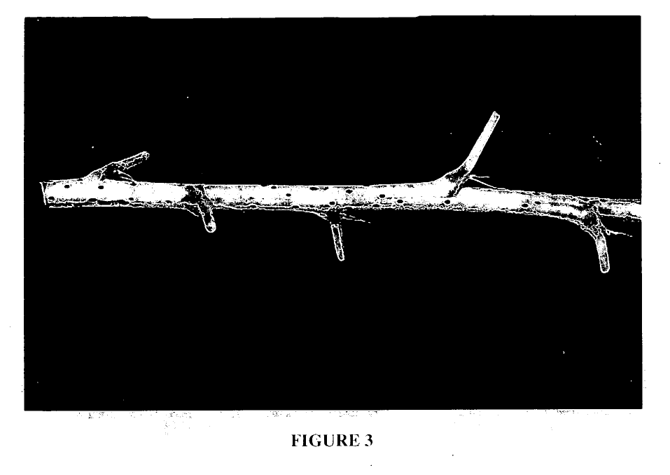

### 5. DETAILED BOTANICAL DESCRIPTION

The following detailed description of the new raspberry cultivar, ‘Driscoll Sevillana’, is based upon recorded observations of plants and fruit grown in Watsonville, Calif. between 2001 and 2002, and is believed to apply to plants of the ‘Driscoll Sevillana’ cultivar grown in similar conditions of soil and climate elsewhere.

Throughout this specification, color names beginning with a small letter signify that the name of the color, as used in common speech, is aptly descriptive. Color data beginning with a capital letter and followed by an alphanumeric code indicates the most similar color designations as provided by The Royal Horticultural Society (RHS) Colour Chart published by The Royal Horticultural Society of London, England. Color designations, color descriptions, and other phenotypical descriptions may deviate from the stated values and descriptions depending upon variation in environmental, seasonal, climatic and cultural conditions.

Table 1 provides information on the plant and fruit characteristics of the new cultivar ‘Driscoll Sevillana’ compared with characteristics of the unpatented raspberry cultivar ‘Heritage’. Comparisons of the cultivars were taken under similar conditions.

The new variety is particularly characterized and distinguished from other cultivars by its large firm fruit with consistent fruit structure and high yield. The fruit color of ‘Driscoll Sevillana’ is a medium red at harvest but darkens after harvest to a deeper color. Fruit of ‘Driscoll Sevillana’ separates easily from the receptacle and is of excellent firmness at harvest. The fruit of ‘Driscoll Sevillana’ is very consistent in size and shape throughout the harvest period.

The primocane and floricane yields of ‘Driscoll Sevillana’ are high relative to the variety ‘Heritage’. ‘Driscoll Sevillana’ is distinguishable from its pollen parent, selection ‘Isabel’, by being a less vigorous plant and producing more uniform and more elongated shape fruit. The new cultivar is distinguished from its seed parent, selection ‘Driscoll Cardinal’, by having larger and less acidic fruit.

### 5.1 DISEASE AND STRESS RESISTANCE

Resistance is unknown to powdery mildew and root rots. Cold tolerance of the new cultivar has not been established. Post harvest fruit rot resistance is good in comparison over many selections and varieties.

TABLE 1
PLANT CHARACTERISTICS OF ‘DRISCOLL SEVILLANA’

| | Driscoll Sevillana | Heritage |
|---|---|---|
| GENERAL | | |
| Plant Size | Small-Med | Large |
| Growth habit | Erect | Erect |
| Productivity | High | Medium |
| Self-fruitfulness | Self-fruitful | Self-fruitful |
| Time of bud burst | Early | Late |
| Primocane fruiting | | |
| Percent of cane Length | 40-60 | 20-40 |
| flowering as Primocane | | |
| Percent of total yield | 60 | 53 |
| Primocanes | | |
| Number of young shoots | Many | Medium |
| Young shoot pigmentation | Medium | Medium |
| Length (cm) | 123 | 195 |
| Time of shoot emergence | Early | Medium |
| Glaucosity (waxy bloom) | Weak | Weak |
| Strength | Medium | Medium |
| Cane cross section (from | Angular | Rounded to angular |
| mid cane of primocane) | | |
| Dormant cane color | 166-A | 166-B |
| Prickles | | |
| Pigmentation | Brown to purple | Green-brownish |
| | | to green |
| Density on young shoots | Medium | Dense |
| Attitude of tip | Downward | Downward |
| Size | Medium | Medium |
| Size: Length (mm) (base | 1.7 | 2 |
| to tip at 1 m height at | | |
| end of harvest) | | |
| Texture | Heavy | Heavy |
| Presence and distribu- | Present, regularly | Present, regularly |
| tion of petioles | distributed | distributed |
| Pubescence on canes | Absent | Absent |
| Internodal distance (cm) | 4.1 | 4.3 |
| (at central 1/3 of cane) | | |
| LEAVES | | |
| Color | | |
| Face | 147-A | 147-A |
| Underside | 148-C | 148-C |
| Relief between veins | Weak | Weak |
| Glossiness | Medium | Medium |
| Petiole | | |
| Length (cm) | 4.4 | 7.0 |
| Pigmentation of upper | Absent | Lightly |
| surface | | |
| Pigmentation of underside | Unpigmented | Unpigmented |
| Stipule orientation | Erect | Erect |
| Arrangement | Compound | Compound |
| Number of leaflets | Sometimes 3, | Sometimes 3, |
| | Sometimes 5 | sometimes 5 |
| Overlapping of lateral | Free to touching | Free to touching |
| leaflets | | |
| Lateral leaflet: length | Very short | Very short |
| to stalklet (lower pair) | | |
| Terminal leaflet | | |
| Length (cm) | 11.5 | 14.8 |
| Width (cm) | 9.5 | 8.8 |
| Shape | Ovate | Ovate |
| Tip | Acuminate | Acuminate |
| Base | Cordate | Rounded |
| Margin | Doubly serrate | Doubly serrate |
| Lateral leaflets | | |
| (basal pair) | | |
| Length (cm) | 10.1 | 13.7 |
| Width | 6.6 | 7.8 |
| Orientation | Opposite | Opposite |
| Shape | Ovate | Ovate |
| Tip | Acuminate | Acuminate |
| Base | Oblique | Oblique |
| Margin | Doubly serrate | Doubly serrate |
| Rachis length between | 2.5-4.5 | 3.7-5.8 |
| terminal leaflet and | | |
| adjacent lateral | | |
| leaflet (cm) | | |
| FLOWERS | | |
| Flowering period | | |
| Primocane | Mid June through | Early July through |
| | Early October | Early October |
| Floricane | Late March through | Mid April through |
| | Late May | Mid June |
| Flower size | Medium | Medium |
| Petal | | |
| Length (cm) | 0.8 | 0.7 |
| Width (cm) | 0.3 | 0.3 |
| Pedicel | | |
| Coloration | Present | Present, strong |
| | | Intensity |
| Length | Medium | Short to medium |
| FRUIT | | |
| Harvest Season | | |
| Primocane | Mid July- | Early August- |
| | early November | early November |
| Floricane | Late April- | Late May- |
| | late June | mid July |
| Color | Medium Red | Medium Red |
| Immature | 180-A | 180-A |
| Maturing | 182-A | 185-B |
| Mature fruit | 185-A | 185-A |
| Glossiness | Medium | Medium |
| Shape | Ovate | Ovate |
| Dimensions | | |
| Size | Medium | Small |
| Length (mm) | 21 | 19 |
| Width (mm) | 21 | 19 |
| Length:width | 1.0 | 1.0 |
| Weight (g/Fruit) | | |
| Primocane | 4.8 | 3.4 |
| Floricane | 4.1 | 2.6 |
| Soluble Solids (%) | 9.0 | 10.5 |
| Titratable acidity | 1.80 | 1.50 |
| (% as citric acid) | | |
| Seeds | | |
| Weight (mg) | 1.74 | 1.62 |
| Number druplets/fruit | 81 | 75 |
| Adherence to plug (1-9) | Medium | Medium |
| Firmness | Medium to firm | Firm |
| Yield | High | Medium |

What is claimed:

1. A new and distinctive cultivar of raspberry plant, substantially as shown and described.
